(19) United States
(12) Patent Application Publication
(10) Pub. No.: US 2008/0254003 A1
(43) Pub. Date: Oct. 16, 2008

(54) Differentiation of Human Embryonic Stem Cells and Cardiomyocytes and Cardiomyocyte Progenitors Derived Therefrom

(76) Inventors: Robert Passier, Ultrecht (NL); Christine Lindsay Mummery, Bilthoven (NL)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/793,858

(22) PCT Filed: Dec. 21, 2005
(86) PCT No.: PCT/AU05/01921

§ 371 Date: Feb. 6, 2008

(30) Foreign Application Priority Data
Dec. 22, 2004 (AU) 2004907294

(51) Int. Cl. A61K 35/12; C12N 5/06; A01K 67/027; A61P 9/00

(52) U.S. Cl. 424/93.7; 435/377; 435/366; 435/404; 800/9

(57) ABSTRACT

The present invention provides a method to improve current culturing methods for the differentiation of cardiomyocytes from hES cells. The method includes culturing the hES cells in the presence of ascorbic acid or a derivative thereof. Preferably the culturing is conducted in serum free conditions. The invention also includes isolated cardiomyocytes and cardiac progenitors differentiated by the methods as well as the use of these cells in methods of treating and preventing cardiac diseases and conditions. Culture media and extracellular media are also provided which include ascorbic acid for the differentiation of hES cells to cardiomyocytes.

### TECHNICAL FIELD

The technical field to which this invention relates is the induction of cardiomyocyte differentiation from human embryonic stem cells.

### BACKGROUND

Cardiomyocytes are thought to be terminally differentiated. Although a small percentage of the cells may have proliferative capacity, it is not sufficient to replace injured or dead cardiomyocytes. Death of cardiomyocytes occurs, for example, when a coronary vessel is occluded by a thrombus and the surrounding cardiomyocytes cannot be supplied with necessary energy sources from other coronary vessels. Loss of functional cardiomyocytes may lead to chronic heart failure.

The proliferative capacity of the cardiomyocytes is not sufficient to regenerate the heart following myocardial injury. Conventional pharmacological therapy for patients with different stages of ischemic heart disease improves cardiac function, survival and quality of life. However, ischemic heart disease is still the most life-threatening disease in western society and alternative therapies will be necessary to improve the clinical outcome for patients with ischemic heart disease further. In recent years, the focus on cell replacement therapy has been intensified, stimulated by the increasing number of potential cell sources for transplantation, such as skeletal myoblasts, adult cardiac stem cells, bone marrow stem cells and embryonic stem cells.

A potential route for restoring “normal” heart function is replacement of injured or dead cardiomyocytes by new functional cardiomyocytes. Human embryonic stem (hES) cells are a potential source of cells for cardiomyocyte replacement. Either spontaneously, or upon induction, differentiation of hES into cardiomyocytes can be achieved.

Embryonic stem cells have a wide differentiation potential. Since the first description of the isolation and characterization of human embryonic stem cells (HESC) from donor blastocysts there have been reports of differentiation of hES to cardiomyocytes. The co-culture of hES with a visceral endoderm-like cell line (END-2), derived from mouse P19 embryonal carcinoma (EC) cells and which resulted in the appearance of beating areas has been demonstrated. The majority (85%) of these hES-derived cardiomyocytes had a ventricle-like phenotype based on morphological and electrophysiological parameters. Contrary to this, others have reported the spontaneous differentiation of hES, cultured as aggregates or embryoid bodies and enhancement of differentiation by demethylating agent 5-aza-deoxycytidine. Between 8 and 70% of the embryoid bodies showed beating areas in these studies, and 2 to 70% of the beating areas consisted of cardiomyocytes. This wide variation in cardiomyocyte differentiation and the relative paucity of quantitative data makes it difficult to compare these in vitro models.

Cardiomyocyte differentiation from hES cells (hES) occurs within 12 days of co-culture with a mouse endoderm-like cell line, END-2. Based on cardiomyocyte phenotype and electrophysiology, the majority of hES-derived cardiomyocytes resemble human fetal ventricular cardiomyocytes. However, the efficiency of cardiomyocyte differentiation from standard co-culture experiments is low.

Hence, there is a need to improve the induction of differentiation of the hES cells to cardiomyocytes to improve the ability to restore cardiac function after myocardial injury.

### SUMMARY OF THE INVENTION

In a first aspect the present invention provides a method for enhancing cardiomyocyte differentiation of a human embryonic stem cell (hES), the method comprising culturing the hES cell in the presence of ascorbic acid, a derivative or functional equivalent thereof.

Preferably the hES cell is co-cultured with another cell which results in cardiomyocyte differentiation in the presence of ascorbic acid, a derivative or functional equivalent thereof.

The present invention provides a method to improve current culturing methods for the differentiation of cardiomyocytes.

Preferably the ascorbic acid used to enhance the cardiomyocyte differentiation is L-ascorbic acid although derivatives and functional equivalents thereof may also be suitable, such as esters and salts of ascorbic acid, or protein bound forms. The concentration of ascorbic acid may vary depending on the conditions for culture.

In a preferred embodiment the culturing conditions are serum-free conditions.

The invention also provides cardiomyocytes and cardiac progenitors derived from the methods. The cardiac progenitors can be identified by their expression of Isl1. Cells derived from the improved method of cardiomyocyte differentiation can be used in transplantation.

The present invention also provides a culture media including ascorbic acid when used in cardiomyocyte differentiation.

### FIGURES

FIG. 1 shows morphology of HESC during END-2 or MEF co-cultures. Morphology of HESC is shown after co-culture with END-2 (A-D) or MEF cells (E-F) for 5 (A, C, E) and 12 days (B, D, F) in the presence (A, B) or in the absence of 20% FCS (C-E). 5× magnifications.

FIG. 2 shows the effect of serum or KSR on the number of beating areas in HESC/END-2 co-cultures. A) Co-cultures were initiated in 12-well plates in different concentrations of FCS and beating areas were counted 12 days later, or were counted from day 8 to 18 (B). C) HESC-END-2 co-cultures were performed in 0% FCS for the first 6 days and in 20% FCS for the next 6 days (0+20(d6)) and vice versa (20+0(d6)). Beating areas were scored on day 12 and compared to 20% FCS and 0% FCS co-cultures. The relative increase as fold-induction with respect to 20% FCS co-cultures is shown. D) Different concentration of KSR is added to HESC-END-2 co-cultures and beating areas are scored on day 12 and compared to 0% FCS co-cultures. Each culture condition was tested in minimally 3 independent experiments. *P<0.05; **P<0.01; ^{a}P<10⁻¹² compared to 20%; ^{###}P<0.001 compared to 20+0(d6)

FIG. 3 shows the effect of serum concentration on the expression of cardiac genes and proteins in HESC/END-2 co-cultures. A) RT-PCR on RNA from 12-day HESC-END-2 co-cultures in 0% FCS or 20% FCS. B) Real-time PCR for α-actinin in 0% FCS (n=3) and 20% FCS (n=2) HESC-END-2 co-cultures using HARP mRNA levels as an internal control. C) Western blot of protein extracts from 12-day HESC-END-2 co-cultures in 0% FCS or 20% FCS and from human fetal cardiomyocytes (HFCM), using antibodies against tropomyosin (TM) and troponin T-C (Trop).

FIG. 4 shows the relationship between beating areas with aαactinin staining and cardiomyocytes after dissociation. A) Beating HESC-END-2 12-day co-cultures from one well are recorded, then fixed and stained for α-actinin (B). Identical areas are indicated by white dashed lines and are labeled from a-e; 5× magnifications. C) 63× magnification of white dashed box of B. D) Dissociated cell of beating areas stained for α-actinin (green) and Topro-3 (blue) (40× magnification). E) Dissociated cells of beating areas stained for Troma-1 (green) and Topro-3 (blue) or α-actinin (red) (F).

FIG. 5 shows the expression of Isl1 in HESC-END-2 co-cultures. A) Real-time PCR for Isl1 in 0% FCS (n=2) and 20% FCS (n=2) 12-day HESC-END-2 co-cultures using HARP mRNA levels as an internal control; *P<0.05. B-D) Isl1 protein localization by immunohistochemistry in 4 μm sections of 12-day beating areas from serum-free HESC-END-2 co-cultures; magnification 20× (B,C) or 40× (D).

FIG. 6 shows the number of cardiomyocytes in 0% and 20% FCS HESC/END-2 co-cultures. A) Beating area of HESC/END-2 12-day co-culture, stained for α-actinin (red) and topro-3 (nucleus, blue) in different planes following confocal scanning (I and I′). Only nuclei surrounded by α-actinin are counted. Examples are given (white arrows); 20× magnification. B) Number of cardiomyocytes from 0% FCS and 20% FCS HESC/END-2 co-cultures are counted and pooled from the different confocal planes. C) Total number of cardiomyocytes from 12-well plate, 12-day HESC/END-2 co-cultures. HESC input represents the estimated number of undifferentiated HESC used per 12-well plate. BA=beating area; CM=cardiomyocytes. D) Co-cultures were initiated in 12-well plates in serum-free HESC-END-2 with or without ascorbic acid (n=6). Beating areas were scored on day 12; *P<0.05.

### DESCRIPTION OF THE INVENTION

In a first aspect the present invention provides a method for enhancing cardiomyocyte differentiation of a human embryonic stem cell (hES), the method comprising culturing the hES cell in the presence of ascorbic acid, a derivative, or functional equivalent thereof.

Ascorbic acid has now been found to assist in the differentiation of cardiomyocytes. In particular, by adding ascorbic acid, a derivative or functional equivalent thereof, cardiomyocyte differentiation may be enhanced over base line differentiation levels. For instance, where cardiomyocyte differentiation of hES cells is spontaneous or is induced under specific cardiomyocyte differentiation inducing conditions, the level of cardiomyocyte differentiation from hES cells to cardiomyocytes or cardiac progenitors can be increased resulting in increased numbers of cardiomyocytes and cardiac progenitors.

It is also conceivable for the present invention to include the use of ascorbic acid, a derivative or functional equivalent thereof to induce cardiomyocyte differentiation from an undifferentiated hES cell population that is capable of differentiation to cardiomyocytes and preferably to direct the differentiation toward a cardiomyocyte lineage.

The addition of ascorbic acid, a derivative or functional equivalent thereof is applicable to any method that is directed to differentiation of hES cells to cardiomyocytes or cardiac progenitors including both directed and spontaneous cardiomyocyte differentiation.

In a preferred embodiment the present invention provides a method for enhancing cardiomyocyte differentiation of a human embryonic stem cell (hES), the method comprising co-culturing the hES cell with another cell or an extracellular medium of the cell culture, under cardiomyocyte or cardiac progenitor differentiating conditions in the presence of ascorbic acid or a derivative thereof. Preferably the cell excretes at least one cardiomyocyte differentiation inducing factor.

The present invention provides a method to improve current culturing methods for the differentiation of cardiomyocytes. Hence “enhancing cardiomyocyte differentiation” can include increasing the number of cardiomyocytes differentiated in a culture compared with a culture that is not enhanced and improving the efficiency of the cardiomyocyte differentiation process. “Enhancing” can also include inducing the cardiomyocyte from an undifferentiated hES cell culture that is capable of cardiomyocyte differentiation.

Preferably the ascorbic acid used to enhance the cardiomyocyte differentiation is L-ascorbic acid although derivatives thereof may also be suitable, such as esters and salts of ascorbic acid, or protein bound forms. The concentration of ascorbic acid may vary depending on the conditions for culture. However, typically, the concentration is about 10⁻³ M to 10⁻⁵ M. Most preferably the concentration is about 10⁻⁴ M. Functional equivalents of the ascorbic acid include compounds that may behave similarly to ascorbic acid.

The ascorbic acid may be introduced at any stage of the culture. Preferably the ascorbic may be present continuously from the initial stage of culture of a hES cell or preferably of a co-culture of the hES cells and more preferably with a cell excreting at least one cardiomyocyte differentiation inducing factor. However, the ascorbic acid may also be introduced when beating areas are visible.

Human embryonic stem cells (HESC or hES cells) can differentiate into cardiomyocytes, but the efficiency of this process is low. Cardiomyocyte differentiation of the hES2 cell line by co-culture with a visceral endoderm-like cell line, END-2, in the presence of 20% fetal calf serum (FCS) has been used previously to induce differentiation. This invention seeks to improve this method and other cardiomyocyte differentiation methods involving hES cells. A serum concentration of 0% to 20% is preferred.

In a most preferred embodiment the culturing conditions are serum-free conditions. The period in which the conditions are serum free are preferably from the time of culture of the hES cells or preferably of the co-culture of the hES cells and more preferably with a cell excreting at least one cardiomyocyte differentiation inducing factor to the time when beating cells are visible. However, the serum free conditions may be introduced at any time after the culture begins.

Introduction of serum free conditions may also be gradual with a preferred reduction of serum over the culture period such as but not limited to a reduction schedule of 20%, 10%, 5%, 2.5% and 0% over the culture period. The concentration may be introduced in a stepwise manner over a range of 20% to 0%. The concentration may be introduced in a stepwise manner so as to introduce the concentrations of 20%, 10%, 5%, 2.5% and 0%.

The period over which cardiomyocyte differentiation is induced may be at least 6 days. Preferably the period is 6 to 12 days. The concentration of the seum may therefore be introduced over this period. For instance some of the period may be in the presence of serum, and the remaining period may be in the absence of serum. Preferably the period is serum free.

There is a striking inverse relationship between cardiomyocyte differentiation and the concentration of FCS during hES2-END-2 co-culture. Applicants have found that the number of beating areas in the co-cultures was increased 24-fold in the absence of FCS, compared to that in the presence of 20% FCS. An additional 40% increase in the number of beating areas was observed when ascorbic acid was added to serum-free co-cultures. The increase in serum-free co-cultures was accompanied by increased mRNA and protein levels of cardiac markers and Isl1, which is a marker for cardiac progenitor cells. The number of beating areas increased up to 12 days after initiation of co-culture of hES2 with END-2 cells. The number of α-actinin positive cardiomyocytes per beating area however did not differ significantly between serum-free co-cultures (503±179; mean±SEM) and 20% FCS co-cultures (312±227). The stimulating effect of serum-free co-culture on cardiomyocyte differentiation of HESC was observed not only in hES2, but also in the hES3 and hES4 cell lines. In order to realize a sufficient number of cardiomyocytes for cell replacement therapy, upscaling cardiomyocyte formation from HESC is essential. The present invention provides a step in this direction and represents a better in vitro model, preferably without interfering factors in serum, for testing other factors that might promote cardiomyocyte differentiation.

The serum-free conditions are most preferred as the serum-free growth itself improves the efficiency of cardiomyocyte differentiation, beating areas being detected earlier and at higher frequency than under standard serum-containing conditions. However, the addition of ascorbic acid can improve or enhance the cardiomyocyte differentiation in substantially serum-free conditions. Hence in those conditions where serum may be present, it is within the spirit of the invention to use ascorbic acid to improve or enhance the cardiomyocyte differentiation.

The contents of WO2005/118784 are herein incorporated by reference as the application describes the culturing of hES cells in serum free media.

The term “inducing differentiation” or “induce differentiation” as used herein is taken to mean causing a stem cell to develop into a specific differentiated cell type as a result of a direct or intentional influence on the stem cell. Influencing factors can include cellular parameters such as ion influx, a pH change and/or extracellular factors, such as secreted proteins, such as but not limited to growth factors and cytokines that regulate and trigger differentiation. It may include culturing the cell to confluence and may be influenced by cell density.

Preferably, the hES cell and any cell preferably providing differentiating factor(s) are co-cultured in vitro. This involves introducing the hES cells preferably to an embryonic cell monolayer produced by proliferation of the embryonic cell in culture. Preferably, the embryonic cell monolayer is grown to substantial confluence and the stem cell is allowed to grow in the presence of extracellular medium of the embryonic cells for a period of time sufficient to induce differentiation of the stem cell to a specific cell type. Alternatively, the stem cell may be allowed to grow in culture containing the extracellular medium of the embryonic cell(s), but not in the presence of the embryonic cell(s). The embryonic cells and stem cells may be separated from each other by a filter or an acellular matrix such as agar.

Preferably for differentiation of stem cells the stem cell can be plated on a monolayer of embryonic cells and allowed to grow in culture to induce differentiation of the stem cell. However, for the purposes of this invention, hES cells may be differentiated to cardiomyocytes and cardiac progenitors by any method to which ascorbic acid can be added to enhance the differentiation.

Conditions for obtaining differentiated embryonic stem cells are typically those which are non-permissive for stem cell renewal, but do not kill stem cells or drive them to differentiate exclusively into extraembryonic lineages. A gradual withdrawal from optimal conditions for stem cell growth favours differentiation of the stem cell to specific cell types. Suitable culture conditions may include the addition of DMSO, retinoic acid, FGFs or BMPs in co-culture which could increase differentiation rate and/or efficiency.

The cell density of the embryonic cell layer typically affects its stability and performance. The embryonic cells are typically confluent. Typically, the embryonic cells are grown to confluence and are then exposed to an agent which prevents further division of the cells, such as mitomycin C. The embryonic monolayer layer is typically established 2 days prior to addition of the stem cell(s). The stem cells are typically dispersed and then introduced to a monolayer of embryonic cells. Preferably, the stem cells and embryonic cells are co-cultured for a period of two to three weeks until a substantial portion of the stem cells have differentiated.

In another aspect of the present invention there is provided a cell culture media for enhancing cardiomyocyte differentiation of a hES cell said culture media comprising ascorbic acid, a derivative or functional equivalent thereof when used for cardiomyocyte differentiation.

The cell culture media delivers ascorbic acid to hES cells for the differentiation to cardiomyocytes and cardiac progenitors. The concentration of the media is preferably of a suitable concentration to deliver ascorbic acid, a derivative or functional equivalent thereof to the hES cells in a range of 10⁻³M to 10⁻⁵M. More preferably the concentration is 10⁻⁴M. Any type of culture media is suitable providing it is suitable for culturing hES cells.

In a preferred embodiment there is provided a cell culture media for enhancing cardiomyocyte differentiation of a hES cell co-cultured with a cell which preferably excretes at least one cardiomyocyte differentiation inducing factor, said culture media comprising ascorbic acid, a derivative or functional equivalent thereof.

Preferably the cell culture media is serum free. However, various concentrations of serum may be tolerated and may range from 20% to 0%. The serum concentrations may also be provided at a concentration selected from the group including 20%, 10%, 5%, 2.5% and 0%.

It is expected that these culture conditions for improved or enhanced cardiomyocyte differentiation will be applicable at least to all hES lines from the same sources as those tested and suggested that these culture conditions for improved cardiomyocyte differentiation are applicable to all hES cell lines and hES cells in general. Furthermore, the fact that these differentiation conditions can be established without fetal calf serum, and thus without the potential presence of animal pathogens, increases the chance that these hES-derived cardiomyocytes are suitable for cardiomyocyte transplantation in patients with heart disease.

The present invention also provides conditions for testing cardiogenic factors. The invention therefore provides a method for testing a factor for cardiogenicity which comprises testing the efficiency of differentiation of hES cells into cardiomyocytes in the presence and absence of the factor. Preferably this method will also comprise culturing the hES cell with a cell which preferably excretes at least one cardiomyocyte differentiation inducing factor or with an extracellular medium therefrom, under conditions that induce differentiation.

Preferably the testing methods adopt serum-free conditions since the Applicants have found that the induction of the differentiation process is enhanced in a serum-free environment in the presence of ascorbic acid, a derivative or functional equivalent thereof.

The invention also provides use of serum free medium containing ascorbic acid, a derivative or functional equivalent thereof in a method of inducing differentiation of hES cells into cardiomyocytes.

Human embryonic stem cells are preferably co-cultured with mouse visceral endoderm (VE)-like cells form beating muscle cells, expressing cardiac specific sarcomeric proteins and ion channels. Direct comparison of electrophysiological responses demonstrates that the majority resemble human fetal ventricular cells in culture, while a minority has an atrial phenotype. This co-culture method permits induction of cardiomyocyte differentiation in hES cells that do not undergo cardiogenesis spontaneously, even at high local cell densities. Both fetal and hES-derived cardiomyocytes in culture are functionally coupled through gap junctions.

Co-culture of pluripotent hES cell lines with END-2 cells induces extensive differentiation to two distinctive cell types from different lineages. One is epithelial and forms large cystic structures staining positively for alpha-fetoprotein and is presumably extraembryonic visceral endoderm; the others are grouped in areas of high local density and beat spontaneously. These beating cells are cardiomyocytes.

The stem cells suitable for use in the present methods comprise both embryonic and adult stem cells and may be derived from a patient's own tissue. This would enhance compatibility of differentiated tissue grafts derived from the stem cells with the patient. In this context it should be noted that hES cells can include adult stem cells derived from a person's own tissue. Human stem cells may be genetically modified prior to use through introduction of genes that may control their state of differentiation prior to, during or after their exposure to the embryonic cell or extracellular medium from an embryonic cell. They may be genetically modified through introduction of vectors expressing a selectable marker under the control of a stem cell specific promoter such as Oct-4. The stem cells may be genetically modified at any stage with a marker so that the marker is carried through to any stage of cultivation. The marker may be used to purify the differentiated or undifferentiated stem cell populations at any stage of cultivation.

Stem cells from which cardiomyocytes are to be derived can be genetically modified to bear mutations in, for example, ion channels (this causes sudden death in humans). Cardiomyocytes derived from these modified stem cells will thus be abnormal and yield a culture model for cardiac ailments associated with defective ion channels. This would be useful for basic research and for testing pharmaceuticals. Likewise, models in culture for other genetically based cardiac diseases could be created. Cardiomyocytes of the present invention can also be used for transplantation and restoration of heart function.

For instance, ischaemic heart disease is the leading cause of morbidity and mortality in the western world. Cardiac ischaemia caused by oxygen deprivation and subsequent oxygen reperfusion initiates irreversible cell damage, eventually leading to widespread cell death and loss of function. Strategies to regenerate damaged cardiac tissue by cardiomyocyte transplantation may prevent or limit post-infarction cardiac failure. The methods of enhancing stem cells to differentiate into cardiomyocytes, as hereinbefore described would be useful for treating such heart diseases. Cardiomyocytes and cardiac progenitors of the invention may also be used in a myocardial infarction model for testing the ability to restore cardiac function.

The human embryonic stem cell may be derived directly from an embryo or from a culture of embryonic stem cells [see for example Reubinoff B E, Pera M F, Fong C Y et al. Embryonic stem cell lines from human blastocysts: somatic differentiation in vitro. Nat Biotechnol 2000; 18: 399-404]. The stem cell may be derived from an embryonic cell line or embryonic tissue. The embryonic stem cells may be cells which have been cultured and maintained in an undifferentiated state.

The hES cell may be an hES cell which does not undergo cardiogenesis spontaneously or alternatively it be an hES cell that does undergo differentiation spontaneously.

It is preferred that the method used to induce the cardiomyocyte differentiation is one involving the co-culture of the hES cell with a cell excreting at least one cardiomyocyte differentiation inducing factor. However, it should be appreciated that the addition of ascorbic acid, a derivative or functional equivalent thereof to any method of culture of hES cells should improve the cardiomyocyte differentiation efficiency. Cells providing cardiomyocyte differentiation inducing factor(s) may be embryonic cells derived from visceral endoderm tissue or visceral endoderm like tissue isolated from an embryo. Preferably, visceral endoderm may be isolated from early postgastrulation embryos, such as mouse embryo (E7.5). Visceral endoderm or visceral endoderm like tissue can be isolated as described in Roelen et al, 1994 Dev. Biol. 166:716-728. Characteristically the visceral endoderm may be identified by expression of alphafetoprotein and cytokeratin (ENDO-A). The embryonic cell may be an embryonal carcinoma cell, preferably one that has visceral endoderm properties. Also included are cells that express endoderm factors or are genetically manipulated to express endoderm factors.

The cardiomyocyte differentiation inducing factor(s) may also be found in extracellular media. Hence it is within the scope of the present invention to use extracellular media derived from a culture of the cell to induce differentiation.

The term “extracellular medium” as used herein is taken to mean conditioned medium produced from growing an embryonic cell as herein described in a medium for a period of time so that extracellular factors, such as secreted proteins, produced by the embryonic cell are present in the conditioned medium. The medium can include components that encourage the growth of the cells, for example basal medium such as Dulbecco's minimum essential medium (DMEM), or Ham's F12 provided in serum free form where serum is a normal component of the medium. END-2 cells are cultured normally in a 1:1 mixture of DMEM with 7.5% FCS, penicillin, streptomycin and 1% non-essential amino acids. In the co-culture with human stem cells the medium is replaced with human embryonic stem cell medium containing 20% or less FCS. In the case of conditioned medium from END-2 cells the conditioned medium may be prepared in serum free form as opposed to the standard 7.5% serum.

In one embodiment, the cell producing cardiomyocyte differentiation inducing factor(s) is a mouse VE-like cell or a cell derived therefrom. Typically, the cell produces a protein excretion profile that is at least substantially as produced by mouse VE-like cells. In a preferred form of this embodiment the cell is an END-2 cell.

The embryonic cell may be derived from a cell line or cells in culture. The embryonic cell may be derived from an embryonic cell line, preferably a cell line with characteristics of visceral endoderm, such as the END-2 cell line (Mummery et al, 1985, Dev Biol. 109:402-410). The END-2 cell line was established by cloning from a culture of P19 EC cells treated as aggregates in suspension (embryoid bodies) with retinoic acid then replated (Mummery et al, 1985, Dev Biol. 109:402-410). The END-2 cell line has characteristics of visceral endoderm (VE), expressing alpha-fetoprotein (AFP) and the cytoskeletal protein ENDO-A.

The contents of WO2003/010303 are referred to and incorporated herein by reference and describes the induction of differentiation of cardiomyocytes in the presence of an embryonic cell.

In another embodiment the cell is a liver parenchymal cell. In a preferred form of this embodiment the liver parenchymal cell is HepG2.

The invention also provides a cardiomyocyte or a cardiac progenitor produced by a method of the invention.

The differentiated cardiomyocyte or cardiac progenitor may express cardiac specific sarcomeric proteins and display chronotropic responses and ion channel expression and function typical of cardiomyocytes.

Preferably, the differentiated cardiomyocyte resembles a human fetal ventricular cell in culture.

In another preferred form the differentiated cardiomyocyte resembles a human fetal atrial cell in culture.

In another preferred form the differentiated cardiomyocyte resembles a human fetal pacemaker cell in culture.

Preferably the cardiac progenitor expresses cardiac markers and in particular, Isl1, a marker for cardiac progenitors. The cells may also express α-actinin. However, a further intermediate cell may express Troma-1. Preferably the cell which expresses Troma-1 is an endoderm-like cell.

The cardiomyocytes of the invention are preferably capable of beating. Cardiomyocytes and the cardiac progenitors, can be fixed and stained with α-actinin antibodies to confirm muscle phenotype. α-troponin, α-tropomysin and α-MHC antibodies also give characteristic muscle staining. Preferably, the cardiomyocytes are fixed according to methods known to those skilled in the art. More preferably, the cardiomyocytes are fixed with paraformaldehyde, preferably with about 2% to about 4% paraformaldehyde. Ion channel characteristics and action potentials of muscle cells can be determined by patch clamp, electrophysiology and RT-PCR.

The present invention provides a plurality of differentiated cardiomyocytes of the invention wherein the differentiated cardiomyocytes are coupled. The coupling may be functional or physical.

In one embodiment the coupling is through gap junctions.

In another embodiment the coupling is through adherens junctions.

In a further embodiment the coupling is electrical.

The present invention also provides a colony of differentiated cardiomyocytes produced by dissociating beating areas from differentiated cardiomyocytes of the invention.

Typically the dissociated cells are replated. Preferably they adopt a two dimensional morphology.

The present invention also provides a model for the study of human cardiomyocytes in culture, comprising differentiated cardiomyocytes or cardiac progenitors of the invention. This model is useful in the development of cardiomyocyte transplantation therapies.

Further, the present invention provides an in vitro system for testing cardiovascular drugs comprising a differentiated cardiomyocyte of the invention.

The present invention also provides a mutated differentiated cardiomyocyte or cardiac progenitor of the invention prepared from a mutant hES cell. It will be recognized that methods for introducing mutations into cells are well known in the art. Mutations encompassed are not only mutations resulting in the loss of a gene or protein but also those causing over expression of a gene or protein.

The present invention provides a method of studying cardiomyocyte differentiation and function (electrophysiology) comprising use of a mutated differentiated cardiomyocyte or cardiac progenitor of the invention.

The present invention provides an in vitro system for testing cardiovascular drugs comprising a mutated differentiated cardiomyocyte of the invention.

The present invention provides an in vitro method for testing cardiovascular drugs comprising using a mutated differentiated cardiomyocyte of the invention as the test cell.

Ion channels play an important role in cardiomyocyte function. If we know which channels are expressed we can make hES cells lacking specific ion channels, and study the effect on cardiac differentiation and function (using electrophysiology). Furthermore, drugs specific for a cardiac ion channel can be tested on cardiomyocyte function (looking at indicators such as action potential, beating frequency, and morphological appearance).

Areas of beating hES-derived cardiomyocytes preferably express ANF. Expression of the α-subunits of the cardiac specific L-type calcium channel (α1c) and the transient outward potassium channel (Kv4.3) are also detected, the expression of Kv4.3 preceding onset of beating by several days. RNA for the delayed rectifier potassium channel KvLQT1 is found in undifferentiated cells, but transcripts disappear during early differentiation and reappear at later stages.

Vital fluorescent staining with ryanodine or antibodies against cell surface α1c ion channels allows differentiated cardiomyocytes of the invention to be identified in mixed cultures. This may provide a means of isolating cardiomyocytes for transplantation without genetic manipulation or compromising their viability.

The present invention also provides differentiated cells produced using methods of the invention that may be used for transplantation, cell therapy or gene therapy. Preferably, the invention provides a differentiated cell produced using methods of the invention that may be used for therapeutic purposes, such as in methods of restoring cardiac function in a subject suffering from a heart disease or condition.

Another aspect of the invention is a method of treating or preventing a cardiac disease or condition. Cardiac disease is typically associated with decreased cardiac function and includes conditions such as, but not limited to, myocardial infarction, cardiac hypertrophy and cardiac arrhythmia. In this aspect of the invention, the method includes introducing an isolated differentiated cardiomyocyte cell of the invention and/or a cell capable of differentiating into a cardiomyocyte cell when treated using a method of the invention into cardiac tissue of a subject. The isolated cardiomyocyte cell is preferably transplanted into damaged cardiac tissue of a subject. More preferably, the method results in the restoration of cardiac function in a subject.

In yet another aspect of the invention there is provided a method of repairing cardiac tissue, the method including
introducing an isolated cardiomyocyte or cardiac progenitor cell of the invention and/or a cell capable of differentiating into a cardiomyocyte cell when treated using a method of the invention into damaged cardiac tissue of a subject.

It is preferred that the subject is suffering from a cardiac disease or condition. In the method of repairing cardiac tissue of the present invention, the isolated cardiomyocyte cell is preferably transplanted into damaged cardiac tissue of a subject. More preferably, the method results in the restoration of cardiac function in a subject.

The present invention preferably also provides a myocardial model for testing the ability of stem cells that have differentiated into cardiomyocytes to restore cardiac function.

The present invention further provides a cell composition including a differentiated cell of the present invention, and a carrier.

The present invention preferably provides a myocardial model for testing the ability of stems cells that have differentiated into cardiomyocytes or cardiac progenitors using methods of the invention to restore cardiac function. In order to test the effectiveness of cardiomyocyte transplantation in vivo, it is important to have a reproducible animal model with a measurable parameter of cardiac function. The parameters used should clearly distinguish control and experimental animals [see for example in Palmen et al. (2001), Cardiovasc. Res. 50, 516-524] so that the effects of transplantation can be adequately determined. PV relationships are a measure of the pumping capacity of the heart and may be used as a read-out of altered cardiac function following transplantation.

A host animal, such as, but not limited to, an immunodeficient mouse may be used as a ‘universal acceptor’ of cardiomyocytes from various sources. The cardiomyocytes are produced by methods of the present invention.

The myocardial model of the present invention is preferably designed to assess the extent of cardiac repair following transplant of cardiomyocytes or suitable progenitors into a suitable host animal. More preferably, the host animal is an immunodeficient animal created as a model of cardiac muscle degeneration following infarct that is used as a universal acceptor of the differentiated cardiomyocytes. This animal can be any species including but not limited to murine, ovine, bovine, canine, porcine and any non-human primates. Parameters used to measure cardiac repair in these animals may include, but are not limited to, electrophysiological characteristic of heart tissue or various heart function. For instance, contractile function may be assessed in terms of volume and pressure changes in a heart. Preferably, ventricular contractile function is assessed. Methods of assessing heart function and cardiac tissue characteristics would involve techniques also known to those skilled in the field.

The present invention further provides a cell composition including a differentiated cell of the present invention, and a carrier. The carrier may be any physiologically acceptable carrier that maintains the cells. It may be PBS or other minimum essential medium known to those skilled in the field. The cell composition of the present invention can be used for biological analysis or medical purposes, such as transplantation.

The cell composition of the present invention can be used in methods of repairing or treating diseases or conditions, such as cardiac disease or where tissue damage has occurred. The treatment may include, but is not limited to, the administration of cells or cell compositions (either as partly or fully differentiated) into patients. These cells or cell compositions would result in reversal of the condition via the restoration of function as previously disclosed above through the use of animal models.

Throughout the description and claims of this specification, the word “comprise” and variations of the word, such as “comprising” and “comprises”, is not intended to exclude other additives, components, integers or steps.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

The present invention will now be more fully described with reference to the accompanying examples and drawings. It should be understood, however that the description following is illustrative only and should not be taken in any way as a restriction on the generality of the invention described above.

### EXAMPLE

### Example 1

### Cardiomyocyte Differentiation in the Presence of Ascorbic Acid

### 1. Materials and Methods

### a) Cell Culture

END-2 cells and HESC lines hES2, hES3 and hES4 cells (passage number between 41-84) were cultured as described previously in Reubinoff B E, Pera M F, Fong C Y et al. Embryonic stem cell lines from human blastocysts: somatic differentiation in vitro. Nat Biotechnol 2000; 18:399-404 and Mummery C, Ward-van Oostwaard D, Doevendans P et al. Differentiation of human embryonic stem cells to cardiomyocytes: role of coculture with visceral endoderm-like cells. Circulation 2003; 107:2733-2740. To initiate co-cultures, END-2 cell cultures, treated for 3 hr with mitomycin C (mit.C; 10 μg/ml), replaced mouse embryonic fibroblasts (MEFs) as feeders for hES cells (Mummery et al (2003) and Mummery C L, van Achterberg T A, van den Eijnden-van Raaij A J et al. Visceral-endoderm-like cell lines induce differentiation of murine P19 embryonal carcinoma cells. Differentiation 1991; 46:51-60). As a control HESC were also grown on MEFs for the same period under the same culture conditions. In standard co-cultures, cells were grown in 12-well plates in DMEM containing L-glutamine, insulin-transferrin-selenium, non-essential amino acids, 90 μM β-mercaptoethanol, penicillin/streptomycin, and 20% FCS (Multicell, Wisent Inc, Canada). Co-cultures were then grown for up to 3 weeks and scored for the presence of areas of beating muscle from 5 days onwards. To study the effect of FCS on cardiomyocyte differentiation concentrations of FCS ranging from 0-20% were compared to standard co-culture conditions. To determine whether the presence or absence of FCS would be critical for cardiomyocyte differentiation throughout the co-culture period, HESC-END-2 co-cultures were conducted in the presence of 20% FCS for the first 6 days and in 0% FCS for the last 6 days, and vice versa. In addition, instead of FCS, various concentrations of knockout serum replacement (KSR) were used during the co-cultures. Finally, serum-free co-culture experiments were also performed in the absence of insulin or insulin-transferrin-selenium (ITS) or in the presence of 10⁻⁴ M ascorbic acid (Sigma, USA).

### b) Primary Human Adult and Fetal Cardiomyocytes.

Primary tissue was obtained during cardiac surgery or following abortion after individual permission using standard informed consent procedures and approval of the ethics committee of the University Medical Center, Utrecht. Fetal cardiomyocytes were isolated from fetal hearts (16-17 weeks) perfused by Langendorff's method and cultured on glass coverslips.

### c) Western Blotting

3 wells of a 12-well plate containing 12-day HESC-END-2 co-cultures, as well as 5-day cultures of human fetal hearts, were washed twice in PBS and collected in 500 μl RIPA-buffer. Protein concentrations were measured by BCA protein assay (Pierce, USA; www.piercenet.com). 50 μg of protein from human fetal hearts and 80 μg of HESC-END2 co-cultures were separated by 10% SDS-PAGE and transferred to PVDF membranes. Blots were incubated with antibodies against sarcomeric tropomyosin (monoclonal, 1:400; Sigma; www.sigmaaldrich.com) and Troponin T-C (goat polyclonal, 1:500; Santa Cruz; www.sccbt.com). Proteins were visualized using ECL.

### d) Immunohistochemistry

HESC-END-2 co-cultures were grown in 12-well plates with 20% or 0% FCS on gelatin-coated coverslips. After 12 days dissected beating areas or whole coverslips were fixed with 2.0% paraformaldehyde for 30 min at room temperature. Fixed beating areas were then embedded in paraffin for immunohistochemistry and 4 μm sections were made. Endogenous peroxidase was blocked in 1.5% H₂O₂ in water, followed by antigen retrieval in citrate buffer. Subsequently, sections were incubated with an antibody against Isl1 (mouse monoclonal 39.4 D5: 1:1000; Developmental Studies Hybridoma Bank, Iowa, USA). Using a secondary goat-anti-mouse antibody (Powervision, ImmunoLogic, the Netherlands) and visualization with 3,3′-diaminobenzidine (Sigma, USA), sections were counterstained with haematoxylin. For immunofluorescence, cells were permeabilized with 0.1% triton X 100 and stained overnight at 4° C. with α-actinin (monoclonal, 1:800; Sigma), α-Troma-1 (rat monoclonal: 1:10, Developmental Studies Hybridoma Bank, Iowa, USA), and used in combination with fluorescent conjugated secondary antibodies (Jackson Immuno Research Laboratories, U.S.A.; Jackson.immuno.com). To visualize nuclei, cells were incubated with Topro-3 (1:1000) in 0.002% Triton.

### e) Cell Counting of α-Actinin Positive Areas

Confocal images (Leica Systems) (10, 20 and 63× objectives) from 2D projected Z-series at 10 μm intervals were made of α-actinin positive areas. Nuclei in α-actinin positive areas were counted. Care was taken to avoid counting the same cells in different planes. Only nuclei surrounded by α-actinin staining in a cardiomyocyte-like striated pattern were counted as positive. All counts were performed double-blind. For immunofluorescent staining of α-actinin on single cells, beating areas were dissected, followed by dissociation as described previously (Mummery et al (2003)). Cells were grown on gelatin-coated coverslips for 7 days.

### f) Reverse Transcriptase PCR

HESC-END-2 co-cultures with 20% or 0% FCS were washed in PBS and RNA from 5 wells pooled using Trizol (Sigma). 500 ng of total RNA was reverse transcribed (Invitrogen; www.invitrogen.com) and used for PCR using Silverstar DNA polymerase (Eurogentec; usa.eurogentec.com). Primer sequences and PCR conditions for α-actinin, ANF, MLC2a, phospholamban and β-actin were described previously (Mummery et al (2003)). The following primer sequences were used.

| | |
|---|---|
| Nkx2.5 (536 bp) | GGTGGAGCTGGAGAAGACAGA |
| | (sense), |
| | CGACGCCGAAGTTCACGAAGT |
| | (anti-sense) |
| GATA-4 (512 bp) | ACCAGGAGGAGCGAGGAGAT |
| | (sense), |
| | GAGAGATGCAGTGTGCTCGT |
| | (anti-sense) |
| α-MHC (542 bp). | GGGGACAGTGGTAAAAGCAA |
| | (sense), |
| | TCCCTGCGTTCCACTATCTT |
| | (anti-sense) |

PCR was performed at 55° C. (annealing temperature) at 1.5 mM MgCl₂ for 30 cycles. Products were analyzed on ethidium bromide-stained 1.5% agarose gel. β-actin was used as RNA input control.

### g) Real-Time Quantitative PCR

Real-time PCR was performed according to standard protocols on a MylQ Real Time PCR detection system (Biorad, USA; www.bio-rad.com) Briefly, 1 μg of total RNA was DNAse treated, and transcribed to cDNA. 10 μl of a 1/10 dilution of cDNA was then added to 12.5 μl of the 2×SYBR green PCR master mix (Applied Biosystems, CA, USA; www.appiedbiosystems.com), and 500 μM of each primer. PCR was performed for α-actinin (sense primer: CTGCTGCTTTGGTGTCAGAG; anti-sense primer: TTCCTATGGGGTCATCCTTG), Isl1 (sense primer: TGATGAAGCAACTCCAGCAG; anti-sense primer: GGACTGGCTACCATGCTGTT) and acidic ribosomal phosphor-protein PO (ARP) (sense primer: CACCATTGAAATCCTGAGTGATGT; antisense primer: TGACCAGCCCAAAGGAGAAG) as an internal control. PCR cycles for α-actinin, Isl1 and HARP were: 3 min. at 95° C., followed by 40 cycles of 15 sec. at 95° C., 30 sec at 62.5° C. and 45 sec at 72° C. The thermal denaturation protocol was run at the end of PCR to determine the number of products. Samples were run on a 2% agarose gel to confirm the correct size of the PCR products. All reactions were run in triplicate. As negative controls PCR was performed on water and on RNA without reverse transcription. The cycle number at which the reaction crossed an arbitrarily placed threshold (CT) was determined for each gene. The relative amount of mRNA levels was determined by 2^{-ΔC}T. Relative gene expression was normalized to ARP expression.

### h) Statistical Analysis

All data are presented as mean±SEM, unless stated otherwise. Statistical significance of differences was calculated using a Student's t-test. Significance was accepted at the level of P<0.05.

### 2. Results

### a) The Effect of Serum on Morphology and the Number of Beating Areas During Co-Culture

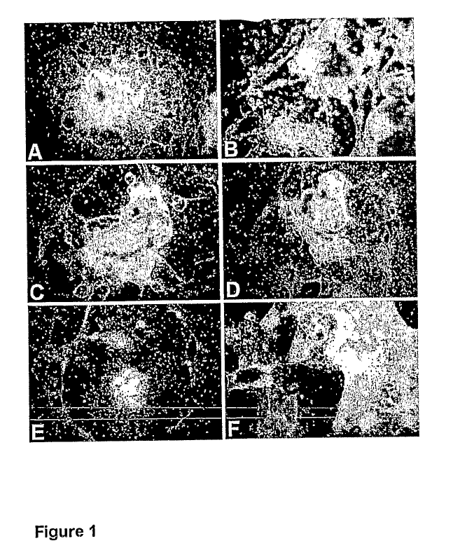

The results described are consistent for all three HESC lines examined (HES-2, -3 and -4). Data are shown from HES-2 cells. To determine the effect of serum on the number of beating areas during co-culture of HESC with END-2 cells, the percentage of serum was decreased to 10%, 5%, 2.5% and 0% from the start at day 1 until the end at day 12 of the co-culture. The number of beating areas in a 12-well co-culture plate was compared with that in the standard 20% FCS co-culture conditions. As shown in FIG. 1, examination of HESC morphology after 5 days in co-culture with 20% FCS demonstrated three-dimensional structures with cells spread out from these structures (FIG. 1A). At 12 days of co-culture this was more evident and strings of differentiating HESC were visible (FIG. 1B). In the absence of serum, the edges of three-dimensional structures were clearer and less spreading of cells was observed (FIGS. 1C and D). HESC cultured on MEF feeders for an additional 12 days in the presence or absence of serum, resulted in a fewer number of cells at day 5 (FIG. 1E), compared to HESC on END-2 cells (FIGS. 1A and C), but not in the formation of three-dimensional structures. At 12 days HESC were more spread out and remained predominantly as a two-dimensional sheet (FIG. 1F).

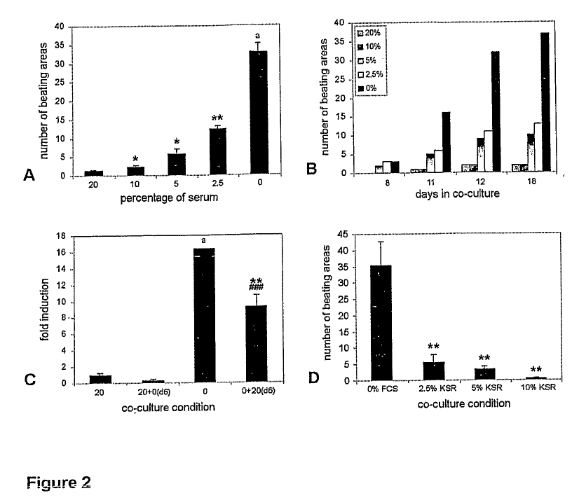

Besides the effect on morphology, a significant increase in the number of beating areas was observed with lower percentages of serum, with a 24-fold up-regulation in its complete absence, when compared to cultures containing 20% FCS (FIG. 2A). On average 1.35±0.26 (n=21) beating areas were observed at day 12 in 20% FCS co-cultures, whereas 32.7±2.3 (n=27) beating areas were observed in 0% FCS co-cultures. Beating areas were normally observed from day 7 onward (occasionally as soon as day 5 or 6) and a linear increase in the number of beating areas was observed until day 12 under all culture conditions. From day 12 onwards additional beating areas appeared, but at a much lower rate (FIG. 2B).

In order to study whether the absence of serum is important throughout the 12-day co-culture period, HESC-END-2 co-culture was initiated in 0% FCS then 20% FCS added at day 6. Conversely, co-cultures were also initiated in the presence of 20% FCS and changed to 0% FCS at day 6. In co-cultures starting in 0% FCS and changed at day 6 for 20% FCS, the number of beating areas decreased to 57% compared to those co-cultures maintained in 0% FCS continuously. However, in the co-cultures in 20% FCS for the first 6 days, the number of beating areas decreased to only 2%, compared to those in 0% FCS continuously (FIG. 2C).

An alternative to serum-free culture is the use of knockout serum replacement (KSR). Various concentrations of KSR were added to HESC-END-2 co-cultures. As shown in FIG. 2D a significant inverse relationship was found between the concentration of KSR in culture medium and the number of beating areas, just as in the FCS supplemented medium. The elimination of insulin or ITS from the serum-free medium during co-culture did not further affect the number of beating areas when compared to serum-free medium alone (data not shown).

### b) Expression of Cardiac Genes and Proteins in 20% and 0% HESC-END-2 Co-Cultures.

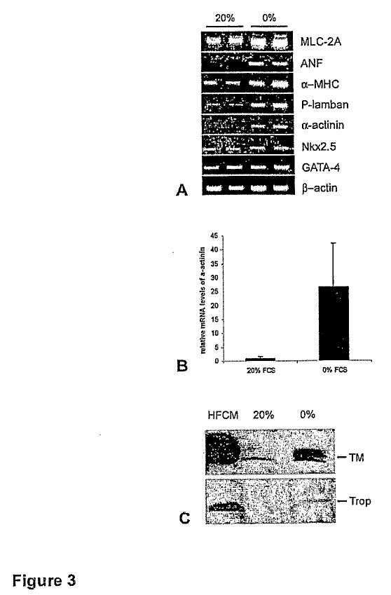

To determine whether the increase in the number of beating areas resulted in a comparable increase in the expression of cardiac genes and proteins, RT-PCR and Western analysis was performed on HESC-END-2 co-cultures in 0% and 20% FCS. A clear increase in the expression for all cardiac genes was observed by RT-PCR in the 0% FCS co-cultures compared with those in 20% FCS (FIG. 3A). Nk×2.5, a homeobox-domain transcription factor, which plays an important role in early cardiac development was slightly up-regulated, whereas the cardiac zinc-finger transcription factor GATA-4, was not changed by 0% FCS compared with 20% FCS co-cultures.

To confirm the results from the semi-quantitative RT-PCR, mRNA levels for α-actinin in 0% and 20% FCS co-cultures were accurately measured by real-time RT-PCR. PCR was performed in triplicate for each sample. As an internal control HARP mRNA levels were determined. Standard deviations were less than 1% for all triplicate reactions. A 27-fold increase in α-actinin mRNA levels was observed in the 0% FCS co-cultures when compared to the 20% FCS co-cultures (FIG. 3B), confirming the results from the RT-PCR.

Increased expression of cardiac structural proteins in 0% FCS co-cultures was confirmed by Western blot analysis. In co-cultures in 20% FCS both tropomyosin and troponin T-C are not detectable or are at the detection limit of the assay, whereas in co-cultures in 0% FCS, clear bands at 36 kDa for tropomyosin and 40 kDa for troponin T-C were observed. As expected an even stronger band at the same molecular weight was observed in protein extracts from human fetal hearts (FIG. 3C).

### c) Characterization of Beating Areas and the Presence of Cardiac Progenitor Cells

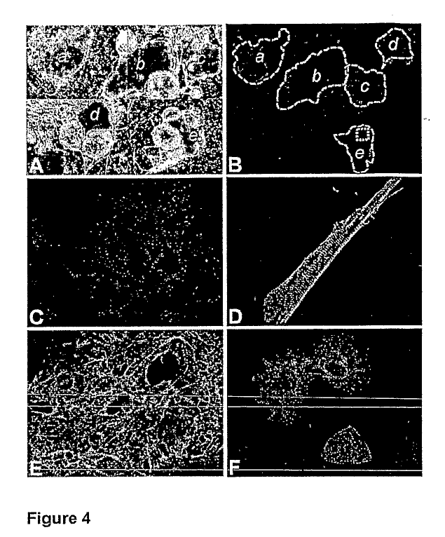

After 12 days, co-cultures in 0% FCS were examined for the presence of beating areas and recorded on video (FIG. 4A). The same samples were then fixed and stained for α-actinin (FIG. 4B) and the films overlayed. All beating areas were also positive for α-actinin and displayed a characteristic cardiomyocyte-like striated pattern (FIG. 4C). No α-actinin positive areas were detected that were not beating before fixation, indicating the high correlation between the number of beating areas and the number of α-actinin-positive areas. Following dissection of beating areas and subsequent dissociation, cells were plated on gelatin-coated dishes, fixed and stained for α-actinin. Between 5 and 20% of the cells were positive for α-actinin (FIG. 4D). The majority of the other cells were positive for Troma-1, which recognizes intermediate cytokeratin 8 and is used as a marker for endoderm (FIG. 4E). By doublestaining immunofluorescence it is clear that α-Troma-1 and α-actinin positive cells do not colocalize (FIG. 4F)

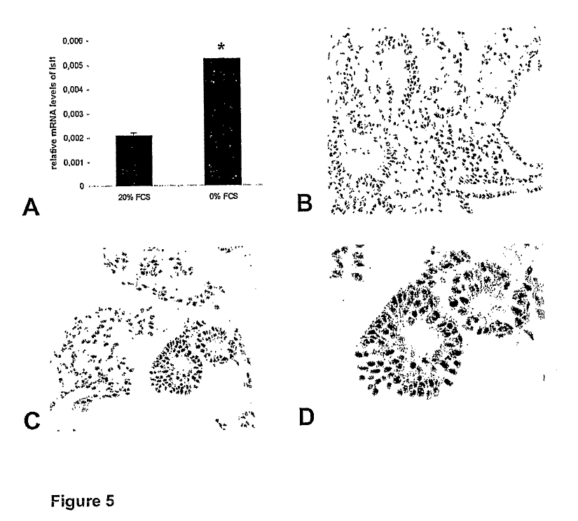

To determine the presence of cardiac progenitor cells in the HESC-END-2 co-cultures we determined the expression of Isl1. By real-time PCR a 2.5 fold increase in the expression of Isl1 was found in serum-free HESC-END-2 co-cultures at day 12, when compared to that of 20% FCS co-cultures (FIG. 5A). By immunohistochemistry we confirmed that nuclear Isl1 protein expression is present in tissue sections of 12 day beating areas (FIGS. 5B-D).

### d) Number of Cardiomyocytes in Co-Cultures

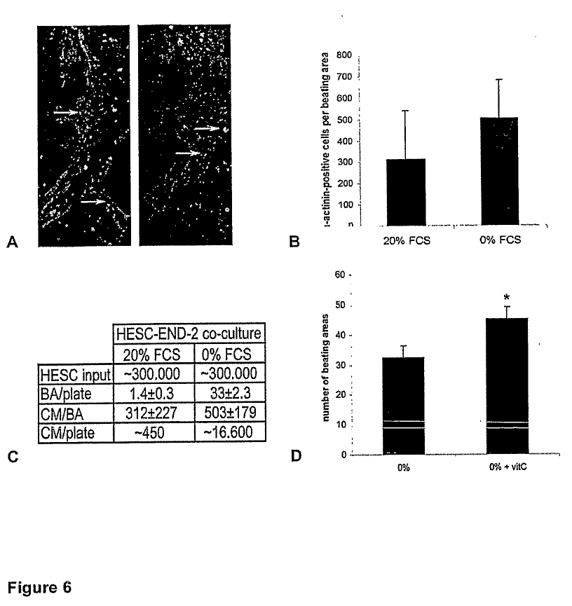

To determine whether the increase in the number of beating areas and the increase in cardiac gene and protein expression was due to the increase of the actual number of cardiomyocytes, α-actinin positive cells with striated sarcomeric patterns were counted. by confocal Z-series. This was considered more informative than FACS analysis, because cells showing striated α-actinin staining could be selectively included. Cells were counted in different planes (FIG. 6A). In co-cultures in 20% FCS the average number of cardiomyocytes per beating area was 312±227 (n=5). The number of cardiomyocytes per beating area in 0% FCS co-cultures was 503±179 (n=15). This was, however, not significantly different and reflects the wide variation in the number of cardiomyocytes per beating area (ranging from 1 to 2500 cells) (FIG. 6B). Based on these numbers, the average total number of cardiomyocytes in a 12 well co-culture plate is therefore approximately 16,600 cells in 0% FCS co-cultures and 450 cells in 20% FCS co-cultures, representing a 39-fold increase in the total number of cardiomyocytes in 0% FCS co-cultures (FIG. 6C).

The serum-free HESC-END-2 co-culture condition represents a better model, without inhibiting factors from serum, for testing other factors for their effect on cardiomyocyte differentiation. Addition of 10⁻⁴ M ascorbic acid to serum-free HESC-END-2 cultures increased the number of beating areas at day 12 by another 40%, when compared to the untreated serum-free co-cultures (FIG. 6D).

HESC can differentiate to cardiomyocytes either spontaneously by growing them as aggregates or embryoid bodies in suspension, or by growing them in co-culture with an endoderm-like cell line, END-2. Efficiency of spontaneous cardiomyocyte differentiation varies between 8% and 70% of the embryoid bodies contracting and reaches a maximum between day 16 and day 30 of differentiation (growth of embryoid bodies in culture followed by plating on gelatin-coated dishes). The percentage of cardiomyocytes in dissected and dissociated beating areas has also been reported to vary widely between 2-70%. Following Percoll gradient centrifugation Xu and colleagues (Xu C, Police S, Rao N et al. Characterization and enrichment of cardiomyocytes derived from human embryonic stem cells. Circ Res 2002; 91:501-508) could obtain a cell fraction, consisting of 70% sMHC positive cells, as determined by immunohistochemistry. Under initial co-culture conditions in the presence of 20% FCS Applicants observed that approximately 16% of the wells of HESC from passage 41-84 contained beating areas. The variation in efficiencies reported for cardiomyocyte formation following spontaneous differentiation has been great. In addition, the lack of standard quantification methods for determining the number of cardiomyocytes, has made it difficult to compare the efficiencies of spontaneous versus induced cardiomyocyte differentiation.

However, an increase in the number of beating areas by reducing FCS in the medium has been described. After 12 days of co-culture, the number of beating areas is 24-fold higher in the absence of FCS compared with 20% FCS. The total number of cardiomyocytes from a 12-well plate is approximately 16,600 cardiomyocytes for co-cultures in 0% FCS, a 39-fold enrichment in the total production of cardiomyocytes per plate compared with serum-containing cultures. The effect of the absence of serum during co-cultures was observed in all HESC lines examined (HES-2, -3 and -4), suggesting a general applicable method for improved cardiomyocyte differentiation.

The permissive effect of serum-free culture conditions on the differentiation of a variety of cell types in culture has been described. Skeletal myoblasts are induced to differentiate by the withdrawal of serum. In addition, undifferentiated neuroblastoma cells form neurites in serum-free medium. In that study replacement of 0.2% FCS/DMEM with serum replacement-2/DMEM, containing, insulin, transferrin and heat-treated bovine serum albumin, resulted in an approximately 4.5-fold increase in the percentage of embryoid bodies that were beating. In addition, the amount of cardiac cMHCα/β, determined by chemiluminescence, was upregulated 6-fold. When the 0.2% FCS/DMEM was replaced with 10% FCS/DMEM after 2 days, neither beating areas nor expression of cMHCα/β were observed. Most protocols for cardiomyocyte differentiation from mouse ES cells use 20% FCS/DMEM. Dependent on the time of presence and the concentration of serum during culture, cardiac differentiation is either stimulated or inhibited. This is in agreement with the present data on HESC: the absence of serum promoted cardiomyocyte differentiation throughout the 12 days of HESC-END-2 co-culture. However, serum-free differentiation conditions clearly had a greater effect on the number of beating areas during the first 6 days of co-culture.

The increase in the number of beating areas in serum-free conditions demonstrates a greater efficiency in cardiomyocyte differentiation. The fact that the expression of cardiac genes and proteins and the number of striated α-actinin-positive cardiomyocytes was significantly increased, largely excluded the explanation that the increase of beating areas is only due to maturation in the organization of sarcomeric contractile units, although it cannot be excluded that increased cardiomyocyte maturation contributes to the effects observed.

Recently it has been described that Isl1, a LIM homeodomain transcription factor is important for cardiac development. Mice lacking Isl1 are missing the outflow tract, right ventricle and much of the atria. Isl1-expressing cells are marking a distinct subset of undifferentiated cardiac progenitor cells. At day 12 in serum-free HESC-END-2 co-cultures, expression of Isl1 mRNA increased 2.5 fold, when compared to serum containing cultures. Also at the protein level, Isl1 could be detected in sections of day 12 beating areas. It is likely that an increased number of cardiac progenitor cells are present in serum-free HESC-END-2 cultures, giving rise to an increased number of beating cardiomyocytes. The finding that in sections of day 12 beating areas Isl1 positive cells are present, suggest that under the right circumstances a further improvement in cardiomyocyte differentiation could be expected.

Of the components of the differentiation medium, insulin or insulin-like growth factors have been shown to have a positive effect on skeletal as well as cardiac differentiation. Co-cultures in serum-free medium without insulin or ITS were performed. The average number of beating areas was not affected by the absence of insulin or ITS; if anything an incidental increase on the number of beating areas was observed (data not shown).

Ascorbic acid has been shown to enhance cardiomyocyte differentiation in the present invention and it is found an additional 40% increase in the number of beating areas in serum-free HESC-END-2 co-cultures in the presence of ascorbic acid.

Previously, it has been shown that the visceral-endoderm-like cell line, END-2, induces mouse P19 embryonal carcinoma (EC), mouse and human embryonic stem cells to aggregate in co-culture and give rise to cultures containing beating areas. For mouse P19 EC cells it has been established that direct contact between the two cell types was not necessary and that a diffusible factor, secreted by the END-2 cells is responsible for the induction of cardiomyocyte formation. Indian hedgehog, secreted from END-2 cells, was shown to be responsible for respecification of prospective neuroectodermal cell fate in mouse epiblast cells along hematopoietic and endothelial lineages. Here it is demonstrated that, in addition to the presence of cardiomyocytes, the majority of the differentiated HESC are Troma-1 positive endodermal-like cells. This suggests that cardiomyocyte differentiation from HESC by END-2 cells could be either directly by END-2 cells, or by HESC-derived endodermal cells.

Accordingly serum-free HESC-END-2 co-culture represents a more defined in vitro model for identifying the cardiomyocyte-inducing activity from END-2 cells and in addition, a more straightforward experimental system for assessing potential cardiogenic factors in addition to ascorbic acid, such as BMPs, FGFs, Wnts and their inhibitors, since there will be no interference from serum-derived modulatory factors.

After dissociation, between 5 and 20% of the cells were α-actinin positive cardiomyocytes. This variation can be attributed to many different factors, such as the size of the beating area, the number of cardiomyocytes per beating area and the accessibility of the beating area (sometimes the beating areas are embedded by non-beating areas). In addition, cell death and attachment during or following dissociation, and time between plating and fixation of dissociated cells play a role in the percentage of cardiomyocytes of the plated dissociated cells (higher proliferation rates of non-cardiomyocytes dilute the percentage of cardiomyocytes present). Therefore selection of cardiomyocytes by FACS, using cell-surface markers, or by genetic manipulation will further stimulate the use of HESC-derived cardiomyocytes for cell-replacement studies.

The higher number of HESC-derived cardiomyocytes in these cultures will not only provide a better in vitro model for understanding cardiac development in humans, but will also facilitate upscale for transplantation studies, to determine whether HESC-derived cardiomyocytes can survive and functionally integrate with host cardiomyocytes, and improve cardiac function in animal models of heart failure. With respect to possible future clinical applications, it is of importance that cardiomyocyte differentiation is feasible in serum-free conditions and thus without the risk of cross transfer with animal pathogens. An alternative for serum, KSR inhibited the number of beating areas, but upon withdrawal the number of beating areas again increased (data not shown). This suggests that maintenance of undifferentiated HESC in the presence of KSR (which would be favorable for future clinical applications), followed by serum-free differentiation cultures, would not affect cardiomyocyte differentiation.

Finally, the invention as hereinbefore described is susceptible to variations, modifications and/or additions other than those specifically described and it is understood that the invention includes all such variations, modifications and/or additions which may be made it is to be understood that various other modifications and/or additions which fall within the scope of the description as hereinbefore described.

1. A method for enhancing cardiomyocyte differentiation of a human embryonic stem cell (hES) the method comprising culturing the hES cell in the presence of ascorbic acid, a derivative, or functional equivalent thereof.

2. A method according to claim 1 wherein the ascorbic acid is present continuously in the culture of the hES cell

3. A method according to claim 1 wherein the ascorbic acid is added to the culture of the hES cell when beating areas are visible.

4. A method according to claim 1 wherein the culture of the hES cell is cultured in the presence of 0% to 20% serum.

5. A method according to claim 1 wherein the hES cell is cultured over a period wherein the serum concentration is reduced in a stepwise manner over a range from approximately 20% to 0%.

6. A method according to claim 1 wherein the culture is serum free.

7. A method according to claim 1 wherein the ascorbic acid, a derivative or functional equivalent thereof is present in the range of 10⁻³ to 10⁻⁵M.

8. A method according to claim 1 wherein the ascorbic acid, a derivative or functional equivalent thereof concentration is 10⁻⁴M.

9. A method according to claim 1 wherein the hES cells is co cultured with another cell that results in cardiomyocyte differentiation.

10. A method according to claim 9 wherein the cell excretes at least one cardiomyocyte differentiation inducing factor.

11. A method according to claim 10 wherein the cell excreting at least one cardiomyocyte differentiation inducing factor is a visceral endoderm or visceral endoderm-like cell.

12. A method according to claim 10 wherein the cell excreting at least one cardiomyocyte differentiation inducing factor is identified by expression of α-fetoprotein and cytokeratin.

13. A method according to claim 10 wherein the cell excreting at least one cardiomyocyte differentiation inducing factor is an END-2 cell line.

14. An isolated cardiomyocyte or cardiac progenitor differentiated from a hES cell prepared by a method according to claim 1.

15. An isolated cardiomyocyte or cardiac progenitor according to claim 14 which expresses the following markers including Isl1, α-actinin, α-troponin, α-tropomysin and α-MHC antibody.

16. An isolated cell population comprising a sub-population of differentiated cells of a cardiomyocyte cell lineage wherein the cardiomyocytes and cardiac progenitors thereof of the cell lineage are differentiated from a hES cell by a method according to claim 1.

17. A cell culture media for enhancing cardiomyocyte differentiation of a hES cell said culture media comprising ascorbic acid, a derivative or functional equivalent thereof when used for cardiomyocyte differentiation.

18. A cell culture media for enhancing cardiomyocyte differentiation in a co-culture of a hES cell with another cell that results in cardiomyocyte differentiation said culture media comprising ascorbic acid, a derivative or functional equivalent thereof when used for cardiomyocyte differentiation.

19. A cell culture media according to claim 18 wherein the cell excretes at least one cardiomyocyte differentiation inducing factor.

20. A cell culture media according to claims 17 or 18 wherein the ascorbic acid is present in the range of 10⁻³ to 10⁻⁵M.

21. A cell culture media according to claims 17 or 18 wherein the ascorbic acid is at a concentration of 10⁻⁴M.

22. A cell culture media according to claims 17 or 18 comprising serum in the range of approximately 20% to 0%.

23. A cell culture media according to claims 17 or 18 which is serum free.

24. A cell culture media according to claims 17 or 18 wherein the media is an extracellular media from a culture of a cell excreting at least one cardiomyocyte differentiation inducing factor.

25. A cell culture media according to claim 24 wherein the cell is a visceral endoderm or visceral endoderm like cell.

26. A cell culture media according to claim 25 wherein the visceral endoderm or visceral endoderm like cell is an END-2 cell line.

27. (canceled)

28. A method of treating or preventing a cardiovascular disease or condition said method comprising transplanting a cardiomyocyte or cardiac progenitor according to claim 14 into a subject in need of the treatment or prevention thereof.

29. A use or method according to claim 28 wherein the cardiovascular disease or condition is selected from the group including myocardial infarction, cardiac hypertrophy and cardiac arrhythmia.

30. (canceled)

31. A cell composition comprising a sub-population of differentiated cells of a cardiomyocyte cell lineage wherein the cell lineage is differentiated from a hES cell by a method according to claim 1 for use in the treatment and prevention of a cardiac disease or condition in a patient.

32. A cell composition according to claim 31 wherein the cell lineage consists of cardiomyocytes or cardiac progenitors differentiated from the hES cell.

33. A cell composition according to claim 31 wherein the cardiac disease or condition is selected from the group including myocardial infarction, cardiac hypertrophy and cardiac arrhythmia.

34. A cell composition comprising a sub-population of differentiated cells of a cardiomyocyte cell lineage wherein the cell lineage is differentiated from a hES cell by a method according to claim 1 for use in repairing damaged cardiac tissue.

35. A cell composition according to claim 34 wherein the cell lineage consists of cardiomyocytes or cardiac progenitors differentiated from the hES cell.

36. A cell composition according to claim 34 wherein the damaged cardiac tissue results from cardiac ischaemia.

37. A method of repairing cardiac tissue said method comprising transplanting a cardiomyocyte or cardiac progenitor according to claim 14 into the cardiac tissue of a subject in need of the repair.

38. A model for testing suitability of a cardiomyocyte cell for cardiac transplantation, said model comprising:
an immunodeficient animal having a measurable parameter of cardiac function wherein said animal is capable of receiving a cardiomyocyte or cardiomyocyte progenitor according to claim 14 by transplantation; and
a means to determine cardiac function of the animal before and after transplantation of the cardiomyocyte.

39. A model according to claim 38 wherein the immunodeficient animal is created as a model of cardiac muscle degeneration following infarct.

40. A model according to claim 38 wherein the parameter of cardiac function is contractile function.
